(19) United States
(12) Patent Application Publication
(10) Pub. No.: US 2024/0033263 A1
(43) Pub. Date: Feb. 1, 2024

(54) COMBINATION OF ALDOSE REDUCTASE INHIBITORS AND PROBENECID FOR THE TREATMENT OF DIABETIC COMPLICATIONS

(71) Applicant: Banavara MYLARI, Lutz, FL (US)

(72) Inventor: Banavara MYLARI, Lutz, FL (US)

(21) Appl. No.: 17/776,718

(22) PCT Filed: Mar. 8, 2022
(86) PCT No.: PCT/US2022/019332

§ 371 Date: Sep. 22, 2022

Related U.S. Application Data
(60) Provisional application No. 63/162,599, filed on Mar. 18, 2021.

(51) Int. Cl. A61K 31/5025; A61K 31/195; A61K 31/502; A61K 31/426

(52) U.S. Cl. CPC A61K 31/5025; A61K 31/195; A61K 31/502; A61K 31/426

(57) ABSTRACT

The subject invention provides pharmaceutical compositions comprising combinations of probenecid or a pharmaceutically acceptable salt thereof, and one or more carboxylic acid aldose reductase inhibitors (ARIs) or pharmaceutically acceptable salts thereof. The subject invention also provides methods of using such combinations to treat mammals, including humans, suffering from diabetic complications such as, diabetic neuropathy, diabetic nephropathy, diabetic retinopathy, diabetic cataracts, diabetic cardiovascular complications, including cardiomyopathy, myocardial infarction, heart failure and atherosclerosis and non-diabetic cardiovascular complications, including myocardial infarction, coronary artery disease, atherosclerotic cardiovascular diseases and heart failure.

### CROSS-REFERENCE TO RELATED APPLICATION

This application claims the benefit of U.S. provisional patent application No. 63/162,599 filed Mar. 18, 2021, which is incorporated by reference herein in its entirety.

### BACKGROUND OF THE INVENTION

Aldose reductase inhibitors (ARIs) function by inhibiting the activity of the enzyme aldose reductase, which is primarily responsible for regulating the reduction of aldoses, such as glucose and galactose, to the corresponding polyols, such as sorbitol and galactitol, in humans and other animals. In this way, unwanted accumulations of galactitol in the lens of galactosemic subjects and of sorbitol in the lens, peripheral nervous cord and kidneys of various diabetic subjects are prevented or reduced.

ARIs have been reported to ameliorate redox imbalance stress in affected tissues (Liang-Jun Y, Animal Model Exp Med. 2018, 1(1) 7-13; S. Vedantham, Cardiovasc. Hematol. Agents Med. Chem. 2012 10(3): 234-240). Oates and Mylari have reviewed the therapeutic uses of ARIs [Aldose reductase inhibitors: Therapeutic implications for diabetic complications. Exp. Opin. Invest. Drugs 8, 2095-2119, (1999)].

Accordingly, ARIs are of medical value for controlling 1) certain diabetic complications, e.g., diabetic neuropathy, diabetic nephropathy, diabetic retinopathy, cataracts, diabetic cardiovascular complications, including cardiomyopathy, myocardial infarction, and heart failure, and 2) non-diabetic cardiovascular complications, including myocardial infarction, coronary artery disease, atherosclerotic cardiovascular disease (R. Ramasamy and I. J. Goldberg, Circ Res. 2010 May 14; 106(9): 1449-1458. S. Vedantham, Arterioscler. Thromb. Vase. Biol. 31(8): 1805-1813).

In particular, Johnson et al. (Diabetes Care 27:448-454, 2004) describes the clinical benefits of zopolrestat ARI in diabetic cardiomyopathy. After 1 year of zopolrestat treatment, there were increases in resting left ventricular ejection fraction (LVEF) (P 0.02), cardiac output (P 0.03), left ventricular stroke volume (P 0.004), and exercise LVEF (P 0.001). In placebo-treated subjects, there were decreases in exercise cardiac output (P 0.03), stroke volume (P 0.02), and end diastolic volume (P 0.04). Exercise LVEF increased with ARI treatment independent of blood pressure, insulin use, or the presence of baseline abnormal heart rate variability. There was no change in resting diastolic filling rates in either group.

ARIs are also useful as therapies in non-diabetic patients for: 1) tissue damage resulting from ischemia (U.S. Pat. No. 6,872,722), 2) reducing tissue damage associated with non-cardiac ischemia (U.S. Pat. No. 6,127,367), and 3) reducing tissue damage associated with ischemia (U.S. Pat. No. 6,872,722).

Probenecid, 4-(dipropylsulfamoyl)benzoic acid, is an uricosuric agent. It is used in the clinic to reduce increased levels of uric acid in blood, for example, in the treatment of gout. It has been in clinical use for >50 years with a limited adverse effect profile. The concomitant oral administration of probenecid increases the mean plasma elimination half-life of certain carboxylic acid (anionic) therapeutic agents such as penicillin when administered either orally or parenterally. The renal excretion of such carboxylic acid drugs is reduced due to probenecid.

Although the clinical significance of this observation has not been fully established, a lower dosage of the above-mentioned carboxylic acid drugs may be required to produce a therapeutic effect and increases in dosage of the drug in question should be made cautiously and in small increments when probenecid is being co-administered. Specific instances of toxicity due to this potential interaction have not been observed to date.

It is noteworthy that probenecid (i.p.) did not alter the plasma profiles and various pharmacokinetic parameters including AUC, clearance, mean residence time, and volume of distribution of N-[4-(benzoylaminophenyl)sulfonyl]glycine (i.v.), an anionic ARI, in Sprague-Dawley rats (Sunkara et al., Brain Res Bull. 81(2-3), 327, 2010). However, in clinical practice probenecid is seldom administered by either i.p or i.v. route.

There is a substantial body of data on the role of probenecid in the cardiovascular arena. Bang et al. (Neurosci Lett. 2007; 425(2):120-125) have found that probenecid is a potent agonist of the transient receptor potential vanilloid 2 (TRPV2) channel. Koch et al. (J Cardiovasc Pharmacol Ther. 2013 May; 18(3): 280-289) have shown that TRPV2 is expressed in the murine heart and, when activated with probenecid, increases cytosolic calcium (Ca2+) concentration, which results in a positive inotropic response both in vitro and in vivo.

Probenecid increases myocardial contractility via increased calcium cycling on a beat-to-beat basis and does not elicit apoptotic or arrhythmic pathways in an animal model (Koch et al. J Mol Cell Cardiol. 2012; 53:134-144). Probenecid has also been found to improve cardiac function in non-diabetic patients with heart failure with reduced ejection fraction (Nathan Robbins et al., J Am Heart Assoc. 2018; 7: e007148. DOI: 10.1161/JAHA.117.007148). Also, probenecid treatment improves outcomes in a novel mouse model of peripartum cardiomyopathy (Evan Onusk et. Al., PLoS One, 2020; 15(3): e0230386).

Interestingly, studies that describe an effect of probenecid on cardiac function were published in 1954 and 1955 and appear to have not been recognized at the time (Koch et al. J Mol Cell Cardiol. 2012, 53(1), 134-144). Nevertheless, U.S. Pat. No. 10,806,711 (Rubinstein et al.) discloses the clinical use of probenecid in treating cardiac dysfunction.

The sodium salt of probenecid (Di Virgilio F., et al. Cell Calcium, 1990, 11, 57) is highly water soluble with a solubility of 77 mg/mL.

AT-001 is in clinical development by Applied Therapeutics for treatment of diabetic cardiomyopathy (American Heart Association 2019 Abstract Mo-2278). Recently Perfetti, Rowell and Shendelman (Applied Therapeutics, Inc. New York, NY USA; European Society for Cardiology 6^{th} World Congress on Acute Heart Failure Athens, Greece, May 25-28, 2019.) have disclosed that the median time of maximum concentration (Tₘₐₓ) of AT-001 was between 1.75-3.38 h in both single oral ascending doses (SAD) and multiple oral ascending doses (MAD) studies in humans and the plasma half-life of AT-001 ranged between 1.74-3.38 hours in both SAD and MAD studies. So, AT-001 would be between approximately 7-12 times shorter lived in human plasma compared to the reported human plasma half-life of zopolrestat (vide infra).

Furthermore, Parfetti and Schendelman (Clinical Assessment of AT-001, an Aldose Reductase Inhibitor in Development for Diabetic Cardiomyopathy: a 28-day proof of concept study) (American Heart Association Scientific Session 2019, Philadelphia, Pennsylvania, November 16-18, 2019) report conducting studies using a BID regimen or a TID regimen, but not a SID regimen.

According to Mylari et al (J. Med. Chem. 1991, 34, 108-122) ARI, zopolrestat shows a sufficiently long plasma half-life in humans (plasma half-life, 27.5 hours), when dosed by the oral route, in a once-a-day dosing regimen to achieve desired efficacy. Johnson et al. (Diabetes Care. 2004, 448-54) conducted a clinical trial with zopolrestat targeted toward treatment of diabetic cardiomyopathy by administering it to patients once-a-day for 6 months.

The ARI Epalrestat (Terashima et al. “Effects of a new aldose reductase inhibitor on various tissue in vitro”. J Pharmacol. Exp. Ther. 229: 226-230 (1984) has a relatively short plasma half-life in humans. Data from six clinical trials were evaluated, and it was determined that epalrestat administered at 50 mg 3 times/day may improve motor and sensory nerve conduction velocity and subjective neuropathy symptoms as compared with baseline and placebo (Pharmacotherapy. 2008 May; 28(5):646-55).

ARIs and probenecid appear to improve reduced ejection fraction and cardiac output in compromised heart patients, seemingly by different biological mechanisms. The former class of compounds has been shown to work both in diabetic animals as well as in diabetic patients. However, to date, the potential of enhanced broader spectrum cardiovascular therapeutic benefits of a combination of carboxylic acid ARIs and probenecid, especially in diabetic patients has not been reported.

Thus, there is a need for developing a combined therapy of ARIs and probenecid for the treatment of, for example, diabetic complications.

### BRIEF SUMMARY OF THE INVENTION

The subject invention provides pharmaceutical compositions comprising an ARI, or a pharmaceutically acceptable salt thereof, and probenecid or a pharmaceutically acceptable salt thereof, formulated with a pharmaceutically acceptable carrier, vehicle, or diluent.

In accordance with the subject invention, probenecid expands the spectrum and potentiates the cardiovascular benefits of carboxylic acid ARIs, for example, AT-001, AT-003, AT-007, zopolrestat, and epalrestat. Concomitantly, probenecid can increase the plasma half-life of the above listed ARIs, especially the relatively shorter-lived carboxylic acid ARIs, such as AT-001 and epalrestat.

The chemical structure of AT-001, AT-003, AT-007, zopolrestat and epalrestat are shown below:

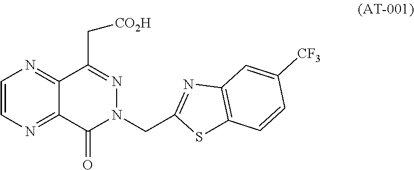 [SMILES: O=C(O)Cc1nn(Cc2nc3cc(C(F)(F)F)ccc3s2)c(=O)c2nccnc12]

### (2-(8-oxo-7-((5-trifluoromethyl)-11H-benzo[d]pyridazin-2-yl) methyl) 8-dihydropyrazin[2,3-d]pyridazine-5-yl)acetic acid])

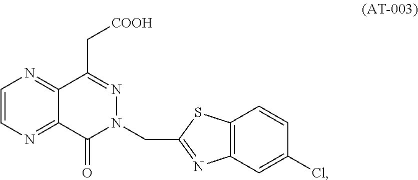 [SMILES: O=C(O)Cc1nn(Cc2nc3cc(Cl)ccc3s2)c(=O)c2nccnc12]

### (2-(8-oxo-7-((5-chloro)-1H-benzo[d]pyridazin-2-yl) methyl) 8-dihydropyrazin[2,3-d]pyridazine-5-yl)acetic acid])

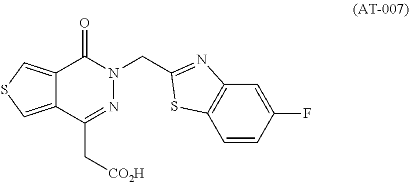 [SMILES: O=C(O)Cc1nn(Cc2nc3cc(F)ccc3s2)c(=O)c2cscc12]

### 2-(3-(benzo[d]thiazol-2-ylmethyl)-4-oxo-3,4-dihydrothieno[3,4-d]pyridazin-1-yl)acetic acid

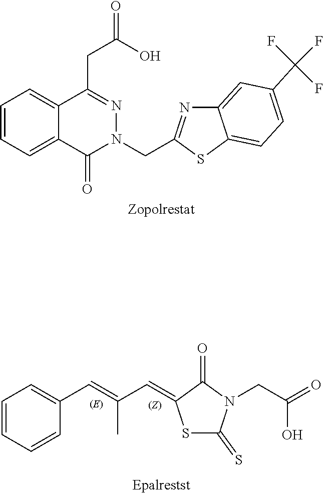 [SMILES: O=C(O)Cc1nn(Cc2nc3cc(C(F)(F)F)ccc3s2)c(=O)c2ccccc12 ; CC(/C=C1\SC(=S)N(CC(=O)O)C1=O)=C\c1ccccc1]

The subject invention also provides methods of treating a diabetic complication in a mammal, including humans, comprising administering to the mammal a pharmaceutical composition as set forth herein. In particular, such diabetic complications include, for example, diabetic neuropathy, diabetic retinopathy and, in preferred embodiments, diabetic cardiomyopathy with abnormalities in LVEF and cardiac output and diabetic myocardial infarction and heart failure.

In one embodiment, the dose of ARI or the pharmaceutically acceptable salt thereof ranges from about 100 to about 3000 mg/day. The dose of probenecid or pharmaceutically acceptable salt thereof ranges from about 100 to about 2000 mg/day.

In a specific embodiment, the ARI, or a pharmaceutically acceptable salt thereof, and probenecid, or a pharmaceutically acceptable salt thereof are administered separately. When administered separately, it is preferable that they are administered concurrently.

In a further embodiment, the ARI, or a pharmaceutically acceptable salt thereof, and probenecid, or pharmaceutically acceptable salt thereof are administered together.

### DETAILED DESCRIPTION OF THE INVENTION

The subject invention provides pharmaceutical compositions comprising an ARI, or a pharmaceutically acceptable salt thereof, and probenecid or a pharmaceutically acceptable salt thereof, formulated with a pharmaceutically acceptable carrier, vehicle, or diluent.

In accordance with the present invention probenecid can be used to advantageously expand the spectrum, and potentiate the clinical benefits, of carboxylic acid ARIs, including AT-001, AT-003, AT-007, zopolrestat, and epalrestat. Concomitantly, probenecid can increase the plasma half-life of the ARIs, especially the relatively shorter-lived carboxylic acid ARIs, such as AT-001 and epalrestat.

The subject invention also provides methods of treating a diabetic complication in a subject, preferably, a mammal, including humans, in need of such treatment, the methods comprising administering to the subject a pharmaceutical composition of the subject invention. In one embodiment, the diabetic complications include, for example, diabetic neuropathy, diabetic retinopathy and, in preferred embodiments, diabetic cardiomyopathy with abnormalities in LVEF and cardiac output, and diabetic myocardial infarction and heart failure.

### Definitions

A “pharmaceutical composition” refers to a mixture of one or more of the compounds described herein, or pharmaceutically acceptable salts, solvates, pro-drugs or hydrates thereof, with other chemical components, such as physiologically acceptable carriers and excipients.

A “pro-drug” or “pro-drug” refers to an agent which is converted into the parent drug in vivo. Pro-drugs are often useful because, in some situations, they are easier to administer than the parent drug. They are bioavailable, for instance, by oral administration whereas the parent drug is either less bioavailable or not bioavailable. The pro-drug also has improved solubility in pharmaceutical compositions over the parent drug. For example, the compound carries protective groups which are split off by hydrolysis in body fluids, e.g., in the bloodstream, thus releasing active compound or is oxidized or reduced in body fluids to release the compound. The term “pro-drug” may apply to such functionalities as, for example, the acid functionalities of the compounds disclosed herein. Pro-drugs may comprise structures wherein an acid group is masked, for example, as an ester or amide.

The term “pharmaceutically acceptable salt” is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, propionic, hexanoic, cyclopentanepropionic, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, magnesium, ammonium, tetraalkylammonium, tetrafluoroborate, ethanolamine, diethanolamine, triethanolamine, N-methylglucamine and the like.

Further salts may include acid addition salts formed with organic acids such as pyruvic acid, malic acid, 3-(4-hydroxybenzoyl)benzoic acid, cinnamic acid, mandelic acid, ethanesulfonic acid, 1,2-ethane-di sulfonic acid, 2-hydroxyethanesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

The terms “treatment” or any grammatical variation thereof (e.g., treat, treating, etc.), as used herein, includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed.

“Pharmaceutically acceptable carrier” refers to a diluent, adjuvant or excipient with which the one or more active agents disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, adjuvant or excipient to facilitate administration of the composition disclosed herein and that is compatible therewith.

Examples of carriers suitable for use in the pharmaceutical compositions are known in the art and such embodiments are within the purview of the invention. The pharmaceutically acceptable carriers and excipients, including, but not limited to, water, oil such as peanut oil, soybean oil, mineral oil, sesame oil and the like, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

### Compositions

In one embodiment, the subject invention provides a pharmaceutical composition comprising an amount of a first compound, an amount of a second compound, and a pharmaceutically acceptable vehicle, carrier, or diluent, wherein the first compound is probenecid, or a pharmaceutically acceptable salt thereof, and the second compound is an aldose reductase inhibitor (ARI) or a pharmaceutically acceptable salt thereof.

In one embodiment, the ARI is selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl) methyl) 8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat, and epalrestat.

The chemical structure of AT-001, AT-003, AT-007, zopolrestat and epalrestat are shown below:

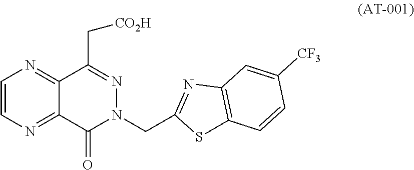 [SMILES: O=C(O)Cc1nn(Cc2nc3cc(C(F)(F)F)ccc3s2)c(=O)c2nccnc12]

### (2-(8-oxo-7-((5-trifluoromethyl)-1H-benzo[d]pyridazin-2-yl) methyl) 8-dihydropyrazin[2,3-d]pyridazine-5-yl)acetic acid])

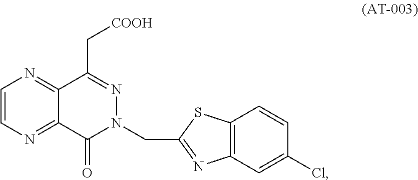 [SMILES: O=C(O)Cc1nn(Cc2nc3cc(Cl)ccc3s2)c(=O)c2nccnc12]

### (2-(8-oxo-7-((5-chloro)-1H-benzo[d]pyridazin-2-yl) methyl) 8-dihydropyrazin[2,3-d]pyridazine-5-yl)acetic acid])

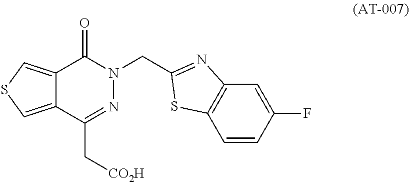 [SMILES: O=C(O)Cc1nn(Cc2nc3cc(F)ccc3s2)c(=O)c2cscc12]

### 2-(3-(benzo[d]thiazol-2-ylmethyl)-4-oxo-3,4-dihydrothieno[3,4-d]pyridazin-1-yl)acetic acid

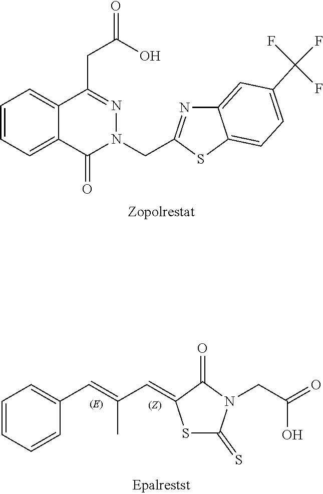 [SMILES: O=C(O)Cc1nn(Cc2nc3cc(C(F)(F)F)ccc3s2)c(=O)c2ccccc12 ; CC(/C=C1\SC(=S)N(CC(=O)O)C1=O)=C\c1ccccc1]

The preparation of AT-001 and AT-003 and their pharmacological and pre-clinical data are given in U.S. Pat. Nos. 8,916,563, 9,650,383 and 10,052,324. The preparation of AT-007 and its pharmacological and pre-clinical data are given in U.S. Pat. No. 10,647,726. The preparation and pharmacologic activity of zopolrestat is described in U.S. Pat. No. 4,939,140. The preparation and pharmacologic activity of epalrestat is described in U.S. Pat. No. 4,831,045.

In one embodiment, the subject invention provides a combined therapy, comprising a combination of two active components: probenecid or a pharmaceutically acceptable salt thereof and an ARI selected from 2-(8-oxo-7-((5-trifluoromethyl)-1H-benzo[d]pyridazin-2-yl) methyl) 8-dihydropyrazin[2,3-d] pyridazine-5-yl)acetic acid, 2-(8-oxo-7-((5-chloro)-1H-benzo[d]pyridazin-2-yl) methyl) 8-dihydropyrazin[2,3-d] pyridazine-5-yl)acetic acid, 2-(3-(benzo[d]thiazol-2-ylmethyl)-4-oxo-3,4-dihydrothieno[3,4-d]pyridazin-1-yl)acetic acid, zopolrestat and epalrestat or a pharmaceutically acceptable salt thereof.

Probenecid is commercially available and is also available by physician prescription. 2-(8-oxo-7-((5-trifluoromethyl)-1H-benzo[d]pyridazin-2-yl) methyl) 8-dihydropyrazin[2,3-d] pyridazine-5-yl) acetic acid and 2-(8-oxo-7-((5-chloro)-1H-benzo[d]pyridazin-2-yl) methyl) 8-dihydropyrazin[2,3-d] pyridazine-5-yl) acetic acid are prepared according to U.S. Pat. No. 8,916,563. 2-(3-(benzo[d]thiazol-2-ylmethyl)-4-oxo-3,4-dihydrothieno[3,4-d]pyridazin-1-yl)acetic acid is prepared according to U.S. Pat. No. 10,647,726. Zopolrestat is prepared according to Mylari et al. (J. Med. Chem. 1991, 34, 108-122). The preparation and pharmacologic activity of epalrestat is described in U.S. Pat. No. 4,831,045.

In one embodiment, the subject invention provides a pharmaceutical composition comprising one or more ARIs, or pharmaceutically acceptable salts thereof, and probenecid, or a pharmaceutically acceptable salt thereof, formulated with a pharmaceutically acceptable carrier, vehicle, or diluent. In one embodiment, the one or more ARIs are selected from AT-001, AT-003, AT-007, zopolrestat and epalrestat.

In one embodiment, the pharmaceutical composition comprises an amount of a first compound, probenecid, or a pharmaceutically acceptable salt thereof and a second compound, ARI, selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl) methyl) 8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat, epalrestat or a pharmaceutically acceptable salt thereof.

In one embodiment, the amount of probenecid or a pharmaceutically acceptable salt thereof is from about 100 to about 2000 mg/day.

In one embodiment, the amount of ARI selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat, epalrestat and pharmaceutically acceptable salts thereof is from about 100 to about 3000 mg/day.

In one embodiment, the composition comprises probenecid or a pharmaceutically acceptable salt thereof, and two ARIs selected from 2-(8-oxo-7-((5-trifluoromethyl)-1H-benzo[d]pyridazin-2-yl) methyl) 8-dihydropyrazin[2,3-d] pyridazine-5-yl)acetic acid, 2-(8-oxo-7-((5-chloro)-1H-benzo[d]pyridazin-2-yl) methyl) 8-dihydropyrazin[2,3-d] pyridazine-5-yl)acetic acid, 2-(3-(benzo[d]thiazol-2-ylmethyl)-4-oxo-3,4-dihydrothieno[3,4-d]pyridazin-1-yl)acetic acid, zopolrestat, epalrestat and pharmaceutically acceptable salts thereof.

In one embodiment, the composition comprises probenecid or a pharmaceutically acceptable salt thereof, and three ARIs selected from 2-(8-oxo-7-((5-trifluoromethyl)-1H-benzo[d]pyridazin-2-yl) methyl) 8-dihydropyrazin[2,3-d] pyridazine-5-yl)acetic acid, 2-(8-oxo-7-((5-chloro)-1H-benzo[d]pyridazin-2-yl) methyl) 8-dihydropyrazin[2,3-d] pyridazine-5-yl)acetic acid, 2-(3-(benzo[d]thiazol-2-ylmethyl)-4-oxo-3,4-dihydrothieno[3,4-d]pyridazin-1-yl)acetic acid, zopolrestat, epalrestat and pharmaceutically acceptable salts thereof.

In one embodiment, the composition comprises probenecid or a pharmaceutically acceptable salt thereof, and four ARIs selected from 2-(8-oxo-7-((5-trifluoromethyl)-1H-benzo[d]pyridazin-2-yl) methyl) 8-dihydropyrazin[2,3-d] pyridazine-5-yl)acetic acid, 2-(8-oxo-7-((5-chloro)-1H-benzo[d]pyridazin-2-yl) methyl) 8-dihydropyrazin[2,3-d] pyridazine-5-yl)acetic acid, 2-(3-(benzo[d]thiazol-2-ylmethyl)-4-oxo-3,4-dihydrothieno[3,4-d]pyridazin-1-yl)acetic acid, zopolrestat, epalrestat and pharmaceutically acceptable salts thereof.

In one embodiment, the composition comprises probenecid or a pharmaceutically acceptable salt thereof, and five ARIs selected from 2-(8-oxo-7-((5-trifluoromethyl)-1H-benzo[d]pyridazin-2-yl) methyl) 8-dihydropyrazin[2,3-d] pyridazine-5-yl)acetic acid, 2-(8-oxo-7-((5-chloro)-1H-benzo[d]pyridazin-2-yl) methyl) 8-dihydropyrazin[2,3-d] pyridazine-5-yl)acetic acid, 2-(3-(benzo[d]thiazol-2-ylmethyl)-4-oxo-3,4-dihydrothieno[3,4-d]pyridazin-1-yl)acetic acid, zopolrestat, epalrestat and pharmaceutically acceptable salts thereof.

In one embodiment, the compositions of the subject invention may contain at least 0.1% of probenecid or a pharmaceutically acceptable salt thereof. The percentage of probenecid or a pharmaceutically acceptable salt thereof presented in such compositions may vary, for example, from about 1% to about 60%, from about 1% to about 50%, from about 1% to about 40%, from about 1% to about 30%, from about 1% to about 20%, from about 1% to about 10%, from about 1% to about 5%, from about 5% to about 50%, from about 5% to about 40%, or from about 10% to about 30% of the weight of a given unit dosage form.

In one embodiment, the compositions of the subject invention may contain at least 0.1% of each of ARIs and pharmaceutically acceptable salts thereof. The percentage of each ARIs and pharmaceutically acceptable salts thereof presented in such compositions may be varied, for example, from about 1% to about 60%, from about 1% to about 50%, from about 1% to about 40%, from about 1% to about 30%, from about 1% to about 20%, from about 1% to about 10%, from about 1% to about 5%, from about 5% to about 50%, from about 5% to about 40%, or from about 10% to about 30% of the weight of a given unit dosage form.

### Formulation and Administration

In one embodiment, the compounds or salts thereof, and compositions of the subjection can be administered via, for example, oral, pulmonary, buccal, suppository, intravenous, intraperitoneal, intranasal, intramuscular or subcutaneous routes. Additional routes of administration are well known to a skilled artisan and such embodiments are within the purview of this invention. The appropriate route of administration depends on the type of condition being treated, the subject being treated, the stage and severity of the condition, etc. A person of ordinary skill in the art can determine an appropriate route of administration based on specific parameters.

The pharmaceutical composition may be, for example, in a form suitable for oral administration as a tablet, capsule, liquid, pill, powder, granules, lozenges (including liquid-filled lozenges), chews, multi- and nano-particulates, gels, ovules, sprays, solution, or sustained release formulation. Such compositions and methods for their preparation may be found, for example, in ‘Remington's Pharmaceutical Sciences’, 19^{th} Edition (Mack Publishing Company, 1995). The pharmaceutical composition may be in unit dosage forms suitable for single administration of precise dosages. The pharmaceutical composition will include a conventional pharmaceutical carrier or excipient and an active compound.

Capsule formulations may be gelatin, soft-gel or solid. Capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants.

Orally administered compositions may contain one or more optional agents such as, for example, sweetening agents such as fructose, aspartame or saccharin; flavoring agents such as peppermint, oil of wintergreen, or cherry; coloring agents; and preservative agents, to provide a pharmaceutically palatable preparation.

Tablet dosage forms typically also include a disintegrant (such as sodium starch glycolate, sodium carboxymethyl cellulose, calcium carboxymethyl cellulose, polyvinylpyrrolidone, methyl cellulose, microcrystalline cellulose, lower alkyl-substituted hydroxypropyl cellulose, starch, pregelatinised starch and sodium alginate) a binder (such as microcrystalline cellulose, gelatin, a sugar, polyethylene glycol, natural and synthetic gums, polyvinylpyrrolidone, pregelatinised starch, hydroxypropyl cellulose and hydroxypropyl methylcellulose) and a lubricant (such as magnesium stearate, calcium stearate, zinc stearate, sodium stearyl fumarate, and mixtures of magnesium stearate with sodium lauryl sulfate). A diluent such as lactose, mannitol, xylitol, dextrose, sucrose, sorbitol, microcrystalline cellulose, starch, and dibasic calcium phosphate dehydrate) may also be present.

The compositions of the invention may also be used in fast-dissolving, fast-disintegrating solid dosage forms such as those described in Expert Opinion in Therapeutic Patents, 11 (6), 981-986, by Liang and Chen (2001).

The composition of the subjection invention may also be administered in a controlled release formulation such as a slow release or a fast release formulation. Such controlled release formulations of the combinations of this invention may be prepared using methods well known to those skilled in the art. The method of administration will be determined by the attendant physician or other person skilled in the art after an evaluation of the subject's condition and requirements.

The composition of the subjection invention may also be administered in parenteral form. For parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, sub-cutaneous and intraperitoneal injection purposes. In this connection, the sterile aqueous media employed are all readily obtainable by standard techniques well-known to those skilled in the art.

The pharmaceutical dosage forms suitable for injection or infusion can include sterile aqueous solutions or dispersions or sterile powders comprising probenecid or a pharmaceutically acceptable salt of said probenecid, and one or more ARIs or salts thereof, which are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by, for example, the formation of liposomes, by the maintenance of the required particle size in the case of dispersions, or by the use of surfactants. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions can be prepared by incorporating probenecid or a pharmaceutically acceptable salt of said probenecid, and one or more ARIs or salts thereof, in the required amount in the appropriate solvent as described herein with various of the other ingredients enumerated herein, preferably followed by filter sterilization. In the case of sterile powders for the preparation of sterile injectable solutions, the preferred methods of preparation are vacuum drying and freeze drying techniques, which yield a powder of probenecid or a pharmaceutically acceptable salt of said probenecid, and/or one or more ARIs or salts thereof, plus any additional desired ingredient present in the previously sterile-filtered solutions.

Pharmaceutical compositions suitable for intra-nasal administration are also encompassed by the present invention. Such intra-nasal compositions probenecid or a pharmaceutically acceptable salt of said probenecid, and one or more ARIs or salts thereof in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising probenecid or a pharmaceutically acceptable salt thereof, and one or more ARIs or pharmaceutically acceptable salts thereof. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of probenecid or a pharmaceutically acceptable salt thereof, and one or more ARIs or pharmaceutically acceptable salts thereof.

Pharmaceutical compositions for administration by inhalation or insufflation can be provided in the form of a dry powder composition, for example, a powder mix of probenecid or a pharmaceutically acceptable salt thereof, and one or more ARIs or pharmaceutically acceptable salts thereof, and a suitable powder base such as lactose or starch. Such powder composition can be provided in unit dosage form, for example, in capsules, cartridges, gelatin packs, or blister packs, from which the powder can be administered with the aid of an inhalator or insufflator.

In one embodiment, a suitable dose of ARIs, or pharmaceutically acceptable salts thereof may range from about 10 to about 5000 mg/day, from about 50 to about 4000 mg/day, from about 100 to about 3000 mg/day, from about 150 to about 2500 mg/day, from about 200 to about 2000 mg/day, from about 250 to about 1500 mg/day, from about 300 to about 1000 mg/day, from about 400 to about 1000 mg/day, from about 500 to about 1000 mg/day, from about 500 to about 800 mg/day, from about 1000 to about 3000 mg/day, from about 1500 to about 3000 mg/day, from about 2000 to about 3000 mg/day, or from about 1000 to about 2000 mg/day.

In one embodiment, a suitable dose of probenecid, or pharmaceutically acceptable salt thereof may range from about 10 to about 3000 mg/day, from about 50 to about 3000 mg/day, from about 100 to about 3000 mg/day, from about 150 to about 2500 mg/day, from about 200 to about 2000 mg/day, from about 250 to about 1500 mg/day, from about 300 to about 1000 mg/day, from about 400 to about 1000 mg/day, from about 500 to about 1000 mg/day, from about 500 to about 800 mg/day, from about 100 to about 2000 mg/day, from about 1000 to about 3000 mg/day, from about 2000 to about 3000 mg/day, or from about 1000 to about 2000 mg/day.

### Methods

The subject invention provides a method for treating a diabetic complication in a subject such as a mammal, including human, comprising administering to said subject a pharmaceutical composition as set forth herein. In particular, such diabetic complications may include, for example, diabetic neuropathy, diabetic retinopathy and especially, diabetic cardiomyopathy with abnormalities in LVEF and cardiac output and diabetic myocardial infarction and heart failure.

In one embodiment, the subject invention provides a method for treating a diabetic complication in a subject such as a mammal, including human, the method comprising administering to said subject an ARI, or a pharmaceutically acceptable salt thereof, and probenecid or a pharmaceutically acceptable salt thereof.

In one embodiment, the subject invention provides a method for treating a diabetic complication in a subject such as a mammal, including human, the method comprising administering to said subject one or more ARIs, or pharmaceutically acceptable salts thereof, and probenecid or a pharmaceutically acceptable salt thereof.

In specific embodiments, the method of the subject invention comprises administering the one or more ARIs or pharmaceutically acceptable salts thereof, and probenecid or pharmaceutically acceptable salt thereof separately. When one or more ARIs or pharmaceutically acceptable salts thereof, and probenecid or pharmaceutically acceptable salt thereof are administered separately, it is preferable that they should be administered concurrently.

In other embodiments, the method of the subject invention comprises administering the one or more ARIs or pharmaceutically acceptable salts thereof, and probenecid or pharmaceutically acceptable salt thereof together.

In certain embodiments, the ARIs or pharmaceutically acceptable salts thereof, and probenecid or pharmaceutically acceptable salt thereof are administered according to this invention simultaneously or sequentially in any order, or as a single pharmaceutical composition comprising one or more ARIs or pharmaceutically acceptable salts thereof, and probenecid or pharmaceutically acceptable salt thereof as described herein.

The methods of this invention include therapeutic treatment of 1) certain diabetic complications, e.g., diabetic neuropathy, diabetic nephropathy, diabetic retinopathy, cataracts, diabetic cardiovascular complications, including cardiomyopathy, myocardial infarction, cardiac ischemia and heart failure, and 2) non-diabetic cardiovascular complications, including myocardial infarction, coronary artery disease, atherosclerotic cardiovascular disease, and heart failure.

In one embodiment, the subject invention provides a method of treating a diabetic complication in a subject such as a mammal (e.g., human), comprising administering to said subject in need of such treatment a pharmaceutical composition of the subject invention.

In one embodiment, the subject invention provides a method of treating a diabetic complication in a subject such as a mammal, comprising administering to said subject in need of such treatment:
(a) an amount from about 100 to about 2000 mg/day of a first compound, wherein the first compound is probenecid or a pharmaceutically acceptable salt thereof; and
(b) an amount from about 100 to about 3000 mg/day of a second compound, wherein the second compound is an ARI or a pharmaceutically acceptable salt thereof, wherein the ARI is selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat, wherein said first compound and said second compound are each optionally and independently administered together with a pharmaceutically vehicle, carrier, or diluent.

In one embodiment, said first and second compounds may be administered via a same or different route.

In one embodiment, provided is a method of treating a diabetic complication in a subject, the method comprising:
administering to the subject an amount of a first compound, wherein the first compound is probenecid or a pharmaceutically acceptable salt thereof, and wherein the amount is, preferably, from about 100 to about 2000 mg/day; and
administering to the subject an amount of a second compound, wherein the second compound is an ARI, a pharmaceutically acceptable salt thereof, or a combination thereof, wherein the ARI is selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat, and wherein the amount is, preferably, from about 100 to about 3000 mg/day,
wherein said first compound and said second compound are each optionally and independently administered together with a pharmaceutically vehicle, carrier, or diluent; and wherein the first compound and the second compound are each independently administered via a same or different route.

In one embodiment, the administration of the first compound may be followed immediately by the administration of the second compound. In some embodiments, an interval exists between the administrations of the first compound and the second compound. The interval may be, for example, at least about 10 min, 20 min, 30 min, 40 min, 50 min, 60 min, 90 min, 120 min, 3 h, 4 h, 5 h, 6 h, 7 h, 8 h, 12 h, 24 h, 36 h, 48 h, 60 h, 72 h, or any in between.

In a preferred embodiment, the diabetic complication is selected from diabetic neuropathy, diabetic nephropathy, diabetic retinopathy, cataracts, diabetic cardiovascular complications. Preferably, the diabetic cardiovascular complication includes, for example, cardiomyopathy, myocardial infarction, cardiac ischemia and heart failure.

In a specific embodiment, the diabetic cardiovascular complications are associated with deficiencies in left ventricular ejection fraction and cardiac output.

In one embodiment, the subject invention provides a method of treating a diabetic complication in a subject such as a mammal, comprising administering to said subject in need of such treatment:
(a) an amount from about 100 to about 3000 mg/day of an ARI or a pharmaceutically acceptable salt thereof, wherein the ARI is selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat; and
(b) an amount from about 100 to about 2000 mg/day of probenecid or a pharmaceutically acceptable salt thereof;
wherein said ARI or a pharmaceutically acceptable salt thereof, and probenecid or a pharmaceutically acceptable salt thereof are each optionally and independently administered together with a pharmaceutically vehicle, carrier, or diluent.

In one embodiment, said ARI or a pharmaceutically acceptable salt thereof, and probenecid or a pharmaceutically acceptable salt thereof may be administered via a same or different route.

In one embodiment, provided is a method of treating a diabetic complication in a subject, the method comprising:
administering to the subject an amount of one or more ARIs, and/or pharmaceutically acceptable salts thereof, wherein the one or more ARIs are selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat, and wherein the amount is, preferably, from about 100 to about 3000 mg/day; and
administering to the subject an amount of probenecid or a pharmaceutically acceptable salt thereof, and wherein the amount is, preferably, from about 100 to about 2000 mg/day;
wherein the one or more ARIs and/or pharmaceutically acceptable salts thereof, and probenecid or a pharmaceutically acceptable salt thereof are each optionally and independently administered together with a pharmaceutically vehicle, carrier, or diluent; and wherein the one or more ARIs and/or pharmaceutically acceptable salts thereof, and probenecid or a pharmaceutically acceptable salt thereof are each independently administered via a same or different route.

In one embodiment, the subject invention provides a method for treating a diabetic complication in a subject, wherein said method comprises administering to a subject in need of such treatment, a pharmaceutical composition comprising:
(a) an amount of a first compound, the first compound being probenecid, or a pharmaceutically acceptable salt thereof; and
(b) an amount of a second compound, the second compound being an ARI or a pharmaceutically acceptable salt thereof, wherein the ARI is selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl)methyl)8-dihydropyrazin[2,3-d]pyridazine-5-yl)acetic acid, 2-(3-((6-fluorobenzo[d]oxazol-2-yl)methyl)-4-oxo-3,4-dihydrothieno[3,4-d]pyridazin-1-yl)acetic acid, zopolrestat, and epalrestat.

In a specific embodiment, the ARI or the pharmaceutically acceptable salt thereof, and/or probenecid, or a pharmaceutically acceptable salt thereof is formulated in a controlled release dosage form.

In one embodiment, the subject has been diagnosed with a diabetic complication. In one embodiment, the subject has been suffering from a diabetic complication such as diabetic neuropathy, diabetic nephropathy, diabetic retinopathy, diabetic cataracts, diabetic cardiovascular complications including cardiomyopathy, myocardial infarction, cardiac ischemia, heart failure and atherosclerosis, and non-diabetic cardiovascular complications including myocardial infarction, coronary artery disease, atherosclerotic cardiovascular diseases and heart failure.

In one embodiment, the subject invention further provides a method of treating a cardiovascular complication in a non-diabetic subject, wherein said method comprises administering, to a subject in need of such treatment:
(a) an amount from 100 to 2000 mg/day of a first compound that is probenecid, or a pharmaceutically acceptable salt of probenecid; and
(b) an amount from 100 to 3000 mg/day of a second compound, which is an ARI selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat or a pharmaceutically acceptable salt of said ARI.

In a further embodiment, the cardiovascular complications include, for example, cardiac ischemia, cardiomyopathy, myocardial infarction, and heart failure.

In one embodiment, the subject invention further provides a method of treating cardiovascular complications in a non-diabetic subject such as a mammal, including a human comprising administering to said subject in need of such treatment a pharmaceutical composition of the subject invention. In one embodiment, the pharmaceutical composition additionally comprises a pharmaceutically acceptable vehicle, carrier, or diluent.

In one embodiment, provided is a method of treating cardiovascular complications in a non-diabetic subject, the method comprising:
administering to the non-diabetic subject an amount of a first compound, wherein the first compound is probenecid or a pharmaceutically acceptable salt thereof, and wherein the amount is, preferably, from about 100 to about 2000 mg/day; and
administering to the non-diabetic subject an amount of a second compound, wherein the second compound is an ARI, a pharmaceutically acceptable salt thereof, or a combination thereof, wherein the ARI is selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat, and wherein the amount is, preferably, from about 100 to about 3000 mg/day,
wherein said first compound and said second compound are each optionally and independently administered together with a pharmaceutically vehicle, carrier, or diluent; and wherein the first compound and the second compound are each independently administered via a same or different route.

Advantageously, the combination of probenecid or a pharmaceutically acceptable salt thereof, and an ARI or a pharmaceutically acceptable salt thereof provides a synergistic effect on diabetic complications and cardiovascular complications.

In one embodiment, the methods comprise administration of a therapeutically effective dosage of an ARI. In some embodiments, the therapeutically effective dosage is at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In one embodiment, the methods comprise administration of a therapeutically effective dosage of probenecid. In some embodiments, the therapeutically effective dosage is at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In one embodiment, the method comprises a single dosage or administration (e.g., as a single injection or deposition). In other embodiments, the method comprises administration once daily, twice daily, three times daily or four times daily to a subject in need thereof for a period of from about 2 to about 28 days, or from about 7 to about 10 days, or from about 7 to about 15 days, or longer.

In some embodiments, the methods comprise chronic administration. In other embodiments, the methods comprise administration over the course of several weeks, months, years or decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

When ranges are used herein, such as for dose ranges, combinations and subcombinations of ranges (e.g., subranges within the disclosed range), specific embodiments therein are intended to be explicitly included.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

All patents, patent applications, provisional applications, and publications referred to or cited herein are incorporated by reference in their entirety, including all figures and tables, to the extent they are not inconsistent with the explicit teachings of this specification.

It should be understood that the embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

1. A pharmaceutical composition comprising an amount of a first compound, an amount of a second compound, and a pharmaceutically acceptable vehicle, carrier, or diluent, wherein the first compound is probenecid, or a pharmaceutically acceptable salt thereof, and the second compound is an aldose reductase inhibitor (ARI) or a pharmaceutically acceptable salt thereof.

2. The pharmaceutical composition according to claim 1, wherein the ARI is selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl) methyl) 8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat, and epalrestat.

3. The pharmaceutical composition according to claim 1, wherein the composition is formulated such that the amount of probenecid or the pharmaceutically acceptable salt thereof is from about 100 to about 2000 mg/day.

4. The pharmaceutical composition according to claim 1, wherein the composition is formulated such that the amount of ARI or the pharmaceutically acceptable salt thereof is from about 100 to about 3000 mg/day.

5. A method for treating a diabetic complication in a mammal, comprising administering to said animal in need of such treatment:
(a) an amount from about 100 to about 2000 mg/day of a first compound, the first compound being probenecid or a pharmaceutically acceptable salt thereof; and
(b) an amount from about 100 to about 3000 mg/day of a second compound, the second compound being an ARI or a pharmaceutically acceptable salt thereof, wherein said first compound and said second compound are each optionally and independently administered together with a pharmaceutically vehicle, carrier, or diluent.

6. The method of claim 5, wherein the ARI is selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat.

7. The method of claim 5, wherein said diabetic complication is diabetic neuropathy, diabetic nephropathy, diabetic retinopathy, cataracts, or diabetic cardiovascular complications.

8. The method of claim 7, wherein the diabetic cardiovascular complication is cardiomyopathy, myocardial infarction, cardiac ischemia or heart failure.

9. The method of claim 7, wherein said diabetic cardiovascular complication is associated with deficiencies in left ventricular ejection fraction and cardiac output.

10. The method of claim 5, wherein the first compound is administered orally, buccally, intravenously, intraperitoneally, intranasally, intramuscularly or subcutaneously.

11. The method of claim 5, wherein the second compound is administered orally, buccally, intravenously, intraperitoneally, intranasally, intramuscularly or subcutaneously.

12. The method of claim 5, wherein the first and second compounds are administered simultaneously or separately.

13. A method of treating a diabetic complication in a subject, wherein said method comprises administering to a subject in need of such treatment, the pharmaceutical composition of claim 1.

14. The method of claim 13, wherein the ARI is selected from 2-(8-oxo-7-((5-trifluoromethyl)-1H-benzo[d]pyridazin-2-yl) methyl) 8-dihydropyrazin[2,3-d]pyridazine-5-yl) acetic acid, 2-(8-oxo-7-((5-chloro)-1H-benzo[d]pyridazin-2-yl)methyl)8-dihydropyrazin[2,3-d]pyridazine-5-yl)acetic acid, 2-(3-((6-fluorobenzo[d]oxazol-2-yl)methyl)-4-oxo-3,4-dihydrothieno[3,4-d]pyridazin-1-yl)acetic acid, zopolrestat, and epalrestat.

15. The method of claim 13, wherein the pharmaceutical composition is administered orally, buccally, intravenously, intraperitoneally, intranasally, intramuscularly or subcutaneously.

16. The method of claim 13, wherein said diabetic complication is diabetic neuropathy, diabetic nephropathy, diabetic retinopathy, cataracts, or diabetic cardiovascular complications.

17. The method of claim 16, wherein the diabetic cardiovascular complication is cardiomyopathy, myocardial infarction, cardiac ischemia or heart failure.

18. The method of claim 16, wherein said diabetic cardiovascular complication is associated with deficiencies in left ventricular ejection fraction and cardiac output.

19. A method of treating a cardiovascular complication in a non-diabetic subject, wherein said method comprises administering, to a subject in need of such treatment:
(a) an amount from 100 to 2000 mg/day of a first compound that is probenecid, or a pharmaceutically acceptable salt of probenecid; and
(b) an amount from 100 to 3000 mg/day of a second compound, which is an ARI selected from 2-(8-oxo-7-((5-trifluoromethyl)-1H-benzo[d]pyridazin-2-yl) methyl) 8-dihydropyrazin[2,3-d] pyridazine-5-yl) acetic acid, 2-(8-oxo-7-((5-chloro)-1H-benzo[d]pyridazin-2-yl)methyl)8-dihydropyrazin[2,3-d]pyridazine-5-yl) acetic acid, 2-(3-((6-fluorobenzo[d]oxazol-2-yl)methyl)-4-oxo-3,4-dihydrothieno[3,4-d]pyridazin-1-yl)acetic acid, zopolrestat and epalrestat or a pharmaceutically acceptable salt of said ARI.

20. The method of claim 19 wherein said cardiovascular complication is cardiac ischemia, cardiomyopathy, myocardial infarction or heart failure.
